(19) United States
(12) Patent Application Publication
(10) Pub. No.: US 2025/0010065 A1
(43) Pub. Date: Jan. 9, 2025

(54) IONTOPHORESIS APPARATUS, ACCOMMODATION MODULE, AND METHOD FOR OPERATING CONTROL CIRCUIT

(71) Applicant: Taiwan Medical Electronics co., ltd., Taipei City (TW)

(72) Inventors: Yu-Pin Cheng, Taipei City (TW); Hua-Lin Hsu, Taipei City (TW); Yue-Feng Wu, Taipei City (TW); Chi-Lin Chen, Taipei City (TW)

(73) Assignee: Taiwan Medical Electronics co., ltd., Taipei City (TW)

(21) Appl. No.: 18/749,196

(22) Filed: Jun. 20, 2024

(30) Foreign Application Priority Data
Jul. 4, 2023 (TW) 112124984

(51) Int. Cl. A61N 1/32; A61N 1/02; A61N 1/04

(52) U.S. Cl. CPC A61N 1/325; A61N 1/025; A61N 1/0448

(57) ABSTRACT

An iontophoresis apparatus including a host, at least two accommodation modules, and a processor is provided. The host includes a housing, a switch mechanism connected to the housing, and at least two host contacts arranged on a side surface of the housing. The accommodation modules each includes a tank body having two side plates arranged oppositely, where a recess is defined in the tank body; an electrode arranged in the recess; and a tank contact arranged on one of the two side plates and electrically connected to the electrode. Two front-end plates of the tank bodies are adapted to be coupled to each other, and the accommodation modules are connected to the host through the tank contacts and the host contacts. The processor is configured to change the value of the current transmitted between the host and the two electrodes in response to that a user triggers the switch mechanism.

### CROSS-REFERENCE TO RELATED APPLICATION

This non-provisional application claims priority under 35 U.S.C. § 119 (a) to patent application No. 112124984 filed in Taiwan, R.O.C. on Jul. 4, 2023, the entire contents of which are hereby incorporated by reference.

### BACKGROUND

### Technical Field

The instant disclosure relates to a therapy device and methods thereof, in particular, to an iontophoresis apparatus and methods thereof.

### Related Art

Iontophoresis (or referred to as ion electrophoresis) therapy is a method by attaching ions generated in ionized liquid onto the skin of a human body to achieve a certain therapeutic purpose. During the therapy, different parts of a user (for example, the user's left and right hands) are placed in two tanks full of liquid, and by electrifying the liquid in the two tanks, the liquid is ionized.

### SUMMARY

However, apparatuses for iontophoresis therapy known to the inventor encounter the same issue, that is, during the therapy, when the user's hands/feet leave the tanks, an electrical shock will be generated to make the user uncomfortable. The possible reason of the generation of the electrical shock may be the sudden release of the charges accumulated at the user's hands/feet (similar to the discharge of a capacitor). It is noted that, although the operation manuals of the apparatuses known to the inventor already suggest that the user should not leave his/her hands/feet arbitrary from the tanks or remind the user the possibility of the occurrence of the electrical shock, the electrical shock issue is still not solved fundamentally.

On the other hand, the apparatus for iontophoresis therapy known to the inventor also fail to provide proper user experiences. For example, for the current loop of the device known to the inventor, the current is transmitted from the user's one hand to user's the other hand through the user's body portion. Therefore, the user has to place his/her two hands/feet in the tanks at the same time for being electrified so as to conduct the therapy. Although the therapy duration may be finished within 20 to 30 minutes, during the therapy, the user cannot do other activities thus the therapy duration experienced by the user will be much longer than the actual therapy duration. Furthermore, upon performing the therapy on the user's two hands, the taller tank often causes that the user has to raise his/her wrists, which is not ergonomic.

Regarding the design concern of the apparatus, it is realized that, a complicated configuration of the apparatus will cause a larger space requirement for the desk or the ground to occupy the apparatus upon performing the therapy and will cause that the apparatus cannot be stored easily.

In view of this, in one embodiment, an iontophoresis apparatus is provided. The iontophoresis apparatus comprises a host, at least two accommodation modules, and a processor. The host comprises a housing, a switch mechanism, and at least two host contacts. The housing has a top surface and a first side surface adjacent to the top surface. The switch mechanism is connected to the housing. The two host contacts are arranged on the first side surface. Each of the two accommodation modules comprises a tank body, an electrode, and a tank contact. The tank body has two side plates arranged oppositely and a front-end plate connected between the two side plates, and a recess is defined in the tank body. The electrode is arranged in the recess of the tank body. The tank contact is arranged on one of the two side plates and electrically connected to the electrode. The two front-end plates of the two tank bodies are adapted to be coupled with each other, and the two accommodation modules are adapted to be coupled to the host through a connection between the two tank contacts and the two host contacts. The processor is configured to change a value of a current transmitted between the host and the two electrodes in response to that the switch mechanism is triggered by a user and the iontophoresis apparatus is in an expanded state.

In some embodiments, the switch mechanism is arranged on the first side surface, a distance between a top surface of the housing and the switch mechanism is less than a distance between the top surface of the housing and each of the two host contacts, and a distance between the switch mechanism and a bottom surface of the host is greater than a height of the two side plates of the tank body.

In some embodiments, the processor is configured to gradually reduce the value of the current transmitted to the two electrodes to a predetermined current value in response to that the processor receives a trigger signal from the switch mechanism.

In some embodiments, the iontophoresis apparatus is adapted to perform therapy on a body portion of a user. When the iontophoresis apparatus is in the expanded state, the body portion of the user is arranged across the two front-end plates of the two tank bodies, so that a portion of the body portion of the user is in the recess of one of the two tank bodies, and the other portion of the body portion of the user in the recess of the other one of the two tank bodies.

In some embodiments, the host further comprises a control circuit, and the processor drives the control circuit to be switched between a first mode and a second mode so as to supply current to the two electrodes. Wherein, the direction of the current passing through one of the two host contacts in the first mode is opposite to the direction of the current passing through the one of the two host contacts in the second mode.

In some embodiments, the iontophoresis apparatus further comprises a conductive medium. The conductive medium comprises a first absorbent section, a nonabsorbent section, and a second absorbent section arranged in sequentially. The conductive medium is adapted to be arranged across the two tank bodies. The first absorbent section and the second absorbent section are adapted to respectively cover the two electrodes of the two tank bodies, and the nonabsorbent section extends along the two front-end plates of the two tank bodies.

In some embodiments, the front-end plate of each of the two tank bodies has a protrusion and an opening, and the two protrusions of the two tank bodies are adapted to be detachably engaged with the two openings of the two tank bodies.

In some embodiments, for each of the two accommodation modules, a bottom surface of the recess of the tank body at least comprises an inclination section, and a height of the inclination section gradually reduces along a direction away from the front-end plate.

In some embodiments, the electrode is fixedly connected to the bottom surface of the recess.

In some embodiments, a height difference is between an upper edge of the front-end plate of the tank body and the bottom surface of the recess, where the height difference is less than 1.5 cm.

In some embodiments, the tank body of each of the two accommodation modules comprises a rear-end plate opposite to the front-end plate and connected between the two side plates, and a height difference is between an upper edge of the rear-end plate and the bottom surface of the recess, where the height difference is less than 3 cm.

In some embodiments, the two host contacts are two first magnetic terminal connectors, the two tank contacts are two second magnetic terminal connectors, and the two host contacts are attached to the two tank contacts through magnetic forces.

In some embodiments, the host further comprises a detection circuit, and the detection circuit is configured to determine a connection state between the tank contacts and the two host contacts.

In some embodiments, a bottom surface of the tank body comprises a footpad.

In some embodiments, the housing of the host further has a second side surface opposite to the first side surface, and the two accommodation modules are separable with each other. In response to that the two tank bodies of the two accommodation modules are stacked with the housing of the host, the first side surface and the second side surface of the host are respectively coplanar with the side plates of the two accommodation modules which are stacked with each other.

In some embodiments, the host further comprises a rotation plate arranged at a corner of the top surface of the housing. The corner of the housing has a stepped structure, and a portion of the rotation plate passes through the stepped structure.

In some embodiments, the housing of the host further comprises a second side surface opposite to the first side surface. The iontophoresis apparatus comprises four host contacts and four accommodation modules. Two of the four host contacts are arranged on the first side surface, and the other two of the four host contacts are arranged on the second side surface. When the iontophoresis apparatus is in the expanded state, the tank contacts of two of the four accommodation modules are connected to the two of the four host contacts which are on the first side surface, and the tank contacts of the other two of the four accommodation modules are connected to the other two of the four host contacts which are on the second side surface.

In another embodiment, an iontophoresis apparatus is provided. The iontophoresis apparatus comprises a host, at least one accommodation tank, and a processor. The host comprises a housing, a switch mechanism, and at least one host contact. The housing has a top surface and a first side surface adjacent to the top surface.

The switch mechanism is connected to the housing. The host contact is arranged on the first side surface. The accommodation tank has two side plates arranged oppositely and an intermediate plate connected between the two side plates. The accommodation tank comprises two accommodation modules and a tank contact. The two accommodation modules are connected to each other through the intermediate plate. Each of the two accommodation modules comprises a tank body and an electrode. A recess is defined in the tank body, and the electrode is arranged in the recess of the tank body. The tank contact is arranged on one of the two side plates of the accommodation tank and electrically connected to the two electrodes. The accommodation tank is adapted to be coupled to the host through a connection between the tank contact and the host contact.

The processor is configured to change a value of a current transmitted between the host and the two electrodes in response to that the switch mechanism is triggered.

In another embodiment, an accommodation module is provided. The accommodation module comprises a tank body, an electrode, and a tank contact. The tank body has two side plates arranged oppositely and a front-end plate connected between the two side plates, where the recess is defined in the tank body. A bottom surface of the recess at least comprises an inclination section, and a height of the inclination section gradually reduces along a direction away from the front-end plate. The electrode is arranged in the recess of the tank body. The tank contact is arranged on one of the two side plates and electrically connected to the electrode.

In some embodiments, the tank contact comprises a magnetic terminal connector.

In some embodiments, a gap between the electrode and the tank body is sealed.

In some embodiments, the accommodation module further comprises a connection structure. The connection structure is configured to allow the accommodation module to be detachably connected to another accommodation module.

In another embodiment, a method for operating control circuit is provided. The method comprises executing a connection state determination operation. The connection state determination operation comprises turning on a first switch; transmitting a test signal from a test signal source to a first host contact through the first switch which is turned on;

and receiving the test signal from the first host contact by using a detection circuit to determine whether the first host contact is coupled to a first tank contact.

In some embodiments, the connection state determination operation further comprises turning on the first switch and a second switch, where the second switch is connected in series to a downstream of the first switch; transmitting another test signal from the test signal source to a second host contact through the first switch and the second switch which are turned on; and receiving the another test signal from the second host contact by using the detection circuit to determine whether the second host contact is coupled to a second tank contact.

In some embodiments, the method further comprises executing an error determination operation before executing the connection state determination operation. The error determination operation comprises turning on a second switch and a third switch, where the second switch and the third switch are sequentially connected in series to a downstream of the first switch; and under a condition that the second switch and the third switch are kept turned on, adjusting the first switch from a turned-off state to a turned-on state, and receiving the test signal from the test signal source by using a current measurement element to determine whether the first switch functions normally.

In another embodiment, a method for executing iontophoresis is provided. The method comprises providing an iontophoresis apparatus mentioned above; supplying a current transmitted between the host and the two electrodes, where the current is adapted to be transmitted between the two electrodes through a body portion of a user arranged across the two tank bodies; determining whether the user triggers the switch mechanism; and changing a value of a current transmitted between the host and the two electrodes in response to that the user triggers the switch mechanism.

### BRIEF DESCRIPTION OF THE DRAWINGS

The disclosure will become more fully understood from the detailed description given herein below for illustration only, and thus not limitative of the disclosure, wherein:

FIG. 1 illustrates a schematic exploded view of components of an iontophoresis apparatus according to an embodiment of the instant disclosure;

FIG. 2 illustrates a schematic right side view of a host according to an embodiment of the instant disclosure;

FIG. 3 illustrates a schematic left side view of a host according to an embodiment of the instant disclosure;

FIG. 4A illustrates a schematic view of an accommodation module according to an embodiment of the instant disclosure;

FIG. 4B illustrates a schematic view of an accommodation tank according to an embodiment of the instant disclosure;

FIG. 5A illustrates a cross-sectional view of two accommodation modules according to an embodiment of the instant disclosure, where the two accommodation modules are assembled with each other;

FIG. 5B illustrates a cross-sectional view of an accommodation tank according to an embodiment of the instant disclosure;

FIG. 6 illustrates a schematic view of an iontophoresis apparatus according to an embodiment of the instant disclosure, where the iontophoresis apparatus is in an expanded state;

FIG. 7 illustrates a right schematic view of an iontophoresis apparatus according to an embodiment of the instant disclosure, where the iontophoresis apparatus is in an expanded state;

FIG. 8A illustrates a schematic view of a conductive medium according to an embodiment of the instant disclosure;

FIG. 8B illustrates a schematic view of a conductive medium according to another embodiment of the instant disclosure;

FIG. 9 illustrates a cross-sectional view of two accommodation modules according to an embodiment of the instant disclosure, where the two accommodation modules are in a used state, a fabric is arranged on the two accommodation modules, and a liquid is filled in the two accommodation modules;

FIG. 10 illustrates a schematic view of an iontophoresis apparatus according to an embodiment of the instant disclosure, where the iontophoresis apparatus is in a stored state;

FIG. 11 illustrates a block diagram of a host according to an embodiment of the instant disclosure;

FIG. 12 illustrates a circuit diagram of a control circuit and a detection circuit of a host according to an embodiment of the instant disclosure;

FIG. 13 illustrates a flowchart of a method for executing iontophoresis according to an embodiment of the instant disclosure; and

FIG. 14 illustrates a schematic view of an iontophoresis apparatus according to an embodiment of the instant disclosure, where the iontophoresis apparatus is in a used state.

### DETAILED DESCRIPTION

To have a clearer understanding of the characteristics, content, advantages of the instant disclosure, and effects that can be achieved by the instant disclosure, the instant disclosure is described in detail below with the accompanying drawings in the form of expressions of embodiments, and the purpose of the drawings used therein are just for illustration. Therefore, the proportion and arrangement relationship of the accompanying drawings should not be interpreted to limit the claim scope of the instant disclosure.

The term “a” or “an” in the instant disclosure is used to describe the elements and components of the instant disclosure. The term is just for the convenience of description and to give the basic concept of the instant disclosure. Such recitation should be understood to include one or at least one, and the singular expression also includes the plural expression unless it is expressly stated otherwise. When the term “a” or “an” is used with the term “comprise” or “include” in the claim, the term “a” or “an” can refer to one or more than one. In addition, the term “or” used in the instant disclosure has the same meaning as “and/or”.

Unless otherwise specified, terms such as “above”, “below”, “up”, “left”, “right”, “down”, “body”, “base”, “vertical”, “horizontal”, “side”, “higher”, “lower portion”, “upper portion”, “top”, “bottom”, or the like for spatial description are used for indicating the direction shown in the figure. It should be understood that, the spatial descriptions used herein are for illustrative purposes only, and that actual implementations of the structures described herein may be spatially configured in any relative orientation, and such limitation does not alter the advantages of embodiments of the instant disclosure.

For example, in the description of some embodiments, providing that one element is “on” another element may encompass situations where the former element is directly on (e.g., in physical contact with) the latter element or that multiple intervening components are between the former element and the latter element.

As used herein, the terms “substantial”, “substantially”, “approximate”, and “approximately” are used to describe and consider for minor changes. It is understood that, when these terms are used in connection with an event or situation, these terms may be referred to both a definite occurrence of the event or situation and a close approximation of the occurrence of the event or situation.

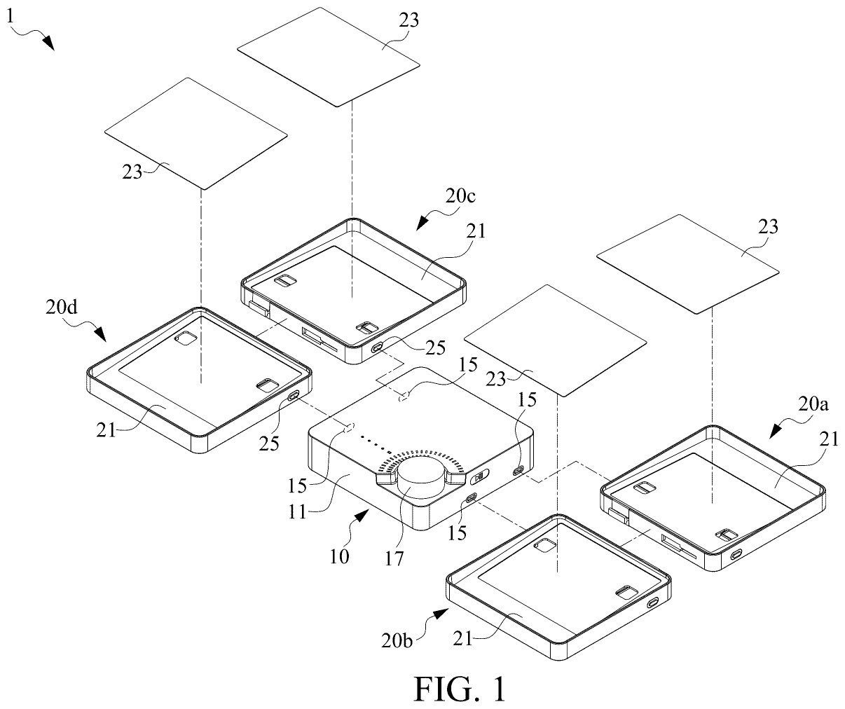

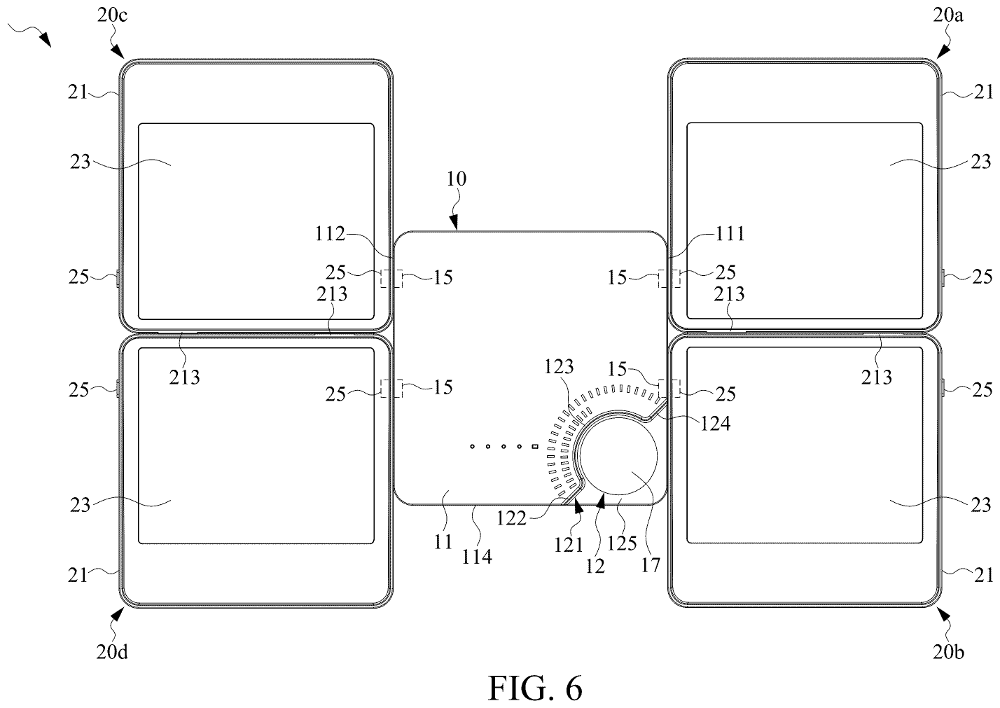

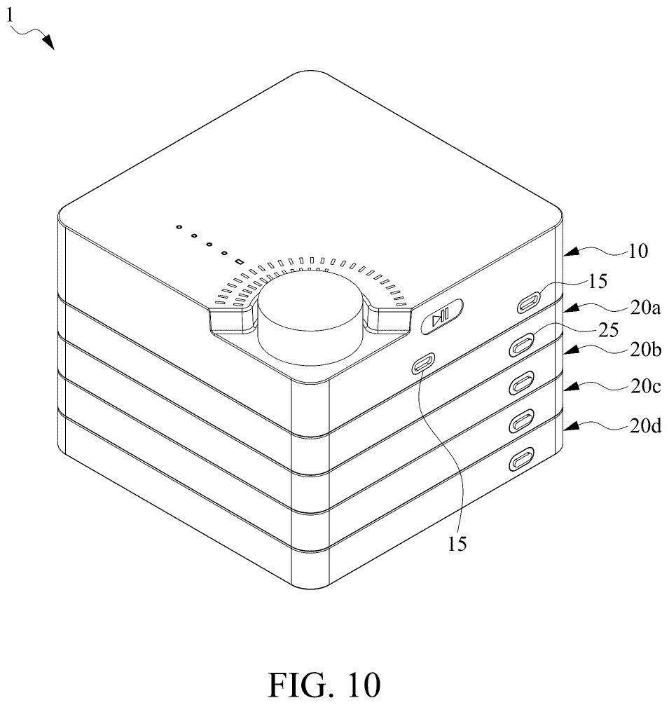

FIG. 1 illustrates a schematic exploded view of components of an iontophoresis apparatus 1 according to an embodiment of the instant disclosure. According to an embodiment of the instant disclosure, the iontophoresis apparatus 1 comprises a host 10 and a plurality of accommodation modules (for example, four accommodation modules 20a, 20b, 20c, 20d). In some embodiments, the four accommodation modules 20a, 20b, 20c, 20d are detachable from the host 10 and thus the state of the iontophoresis apparatus 1 can be changed between an expanded state (as shown in FIG. 6) and a stored state (as shown in FIG. 10). In the expanded state, tank contacts 25 on the four accommodation modules 20a, 20b, 20c, 20d are connected to host contacts 15 at a side surface of the host 10, so that the four accommodation modules 20a, 20b, 20c, 20d are connected to the host 10. In this embodiment, in the stored state, the four accommodation modules 20a, 20b, 20c, 20d may be stacked below the host 10. In other embodiments, the two accommodation modules 20a, 20b together form a set, the two accommodation modules 20c, 20d together form a set, the set of the two accommodation modules 20a, 20b is stacked with the set of the two accommodation modules 20c, 20d (not shown), and the host 10 is stacked on one of the accommodation modules 20a, 20b, 20c, 20d. In the following paragraphs, the structural configuration of the host 10 and the accommodation modules 20a, 20b, 20c, 20d according to some embodiments of the instant disclosure are described.

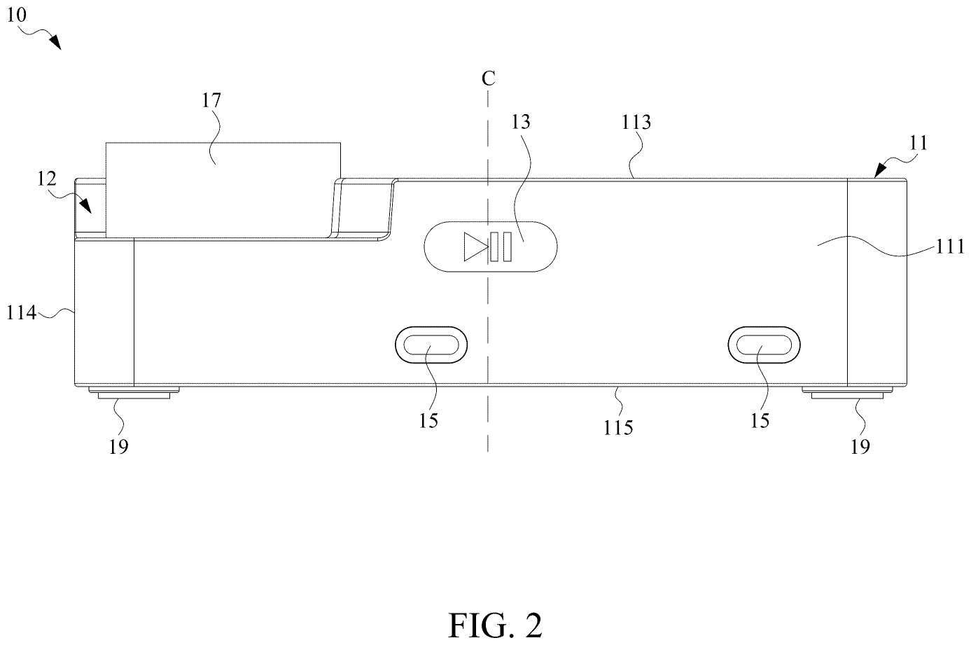

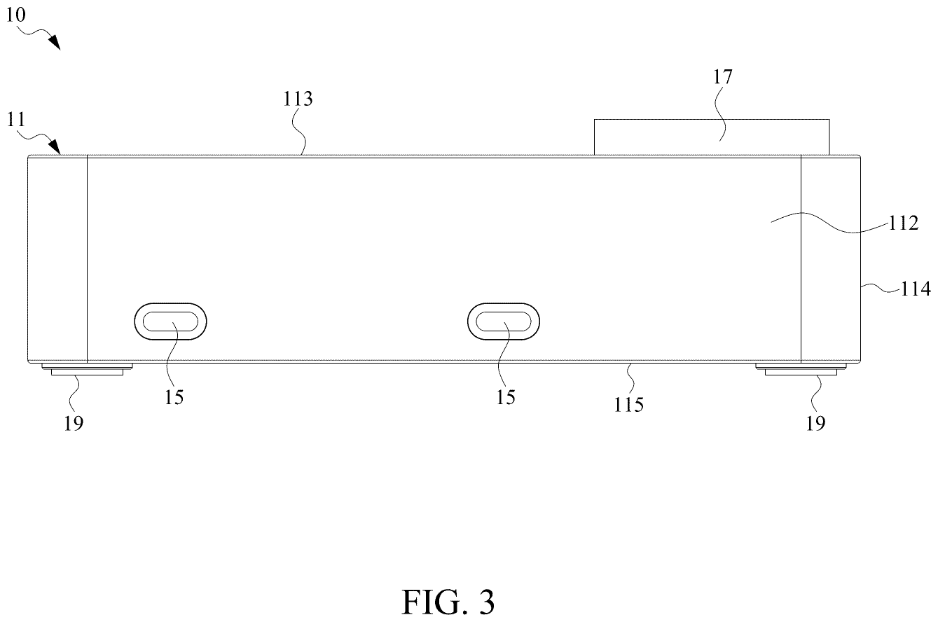

FIG. 2 illustrates a schematic right side view of a host 10 according to an embodiment of the instant disclosure. FIG. 3 illustrates a schematic left side view of a host 10 according to an embodiment of the instant disclosure. As shown in FIG. 1 to FIG. 3, in some embodiments, the host 10 comprises a housing 11, a switch mechanism 13, a plurality of host contacts 15, a rotation plate 17, and a plurality of footpads 19. In some embodiments, the housing 11 is substantially formed as a hexahedron and comprises a first side surface 111, a second side surface 112, a top surface 113, and a front side surface 114. The first side surface 111 and the second side surface 112 are at left and right sides of the housing 11, the top surface 113 is at the top of the housing 11 and connected between the first side surface 111 and the second side surface 112, and the front side surface 114 is at the front of the housing 11 and connected between the first side surface 111 and the second side surface 112.

The switch mechanism 13 is configured to be pressed, touched, or approached by the user to control the current output by the host 10. In some embodiments, as shown in FIG. 2, the switch mechanism 13 is arranged on the first side surface 111 of the housing 11. The switch mechanism 13 may comprise a mechanical button, for example, a membrane switch button or a micro-switch button. Alternatively, in some other embodiments, the switch mechanism 13 may comprise a non-mechanical switch button, for example, a capacitive touch sensor, a resistive touch sensor, a spring touch sensor, a pressure touch sensor, or an optical sensor (for example, the optical sensor may be triggered by shielding the light incident to the sensor). In the case that the switch mechanism 13 is a non-mechanical switch button, the housing 11 can accommodate the switch mechanism 13 without being provided with an opening. Therefore, moisture can be prevented from entering the interior of the housing 11. In some embodiments, the switch mechanism 13 comprises a concave or convex or patterned region on the housing 11 and comprises the non-mechanical switch button correspondingly configured adjacent to the region. The region is adapted to instruct the user to press, touch, or approach thereof. For example, the spring touch sensor is arranged on an inner side of the first side surface 111 of the housing 11, and the outer side of the first side surface 111 corresponding to the spring touch sensor is a region with patterned indication.

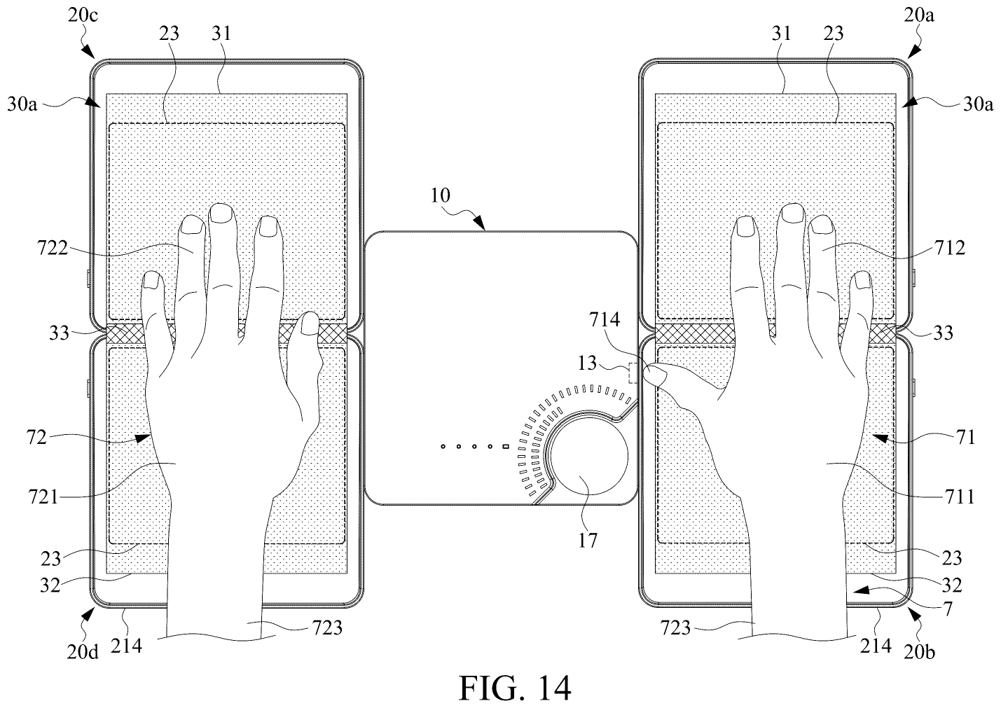

The host contacts 15 are adapted to output the current supplied by the host 10 or to receive the current input from the accommodation modules 20a, 20b, 20c, 20d. The host contact 15 may be referred to a terminal connector comprising one or more electrical contacts (pins) or may be referred to the electrical contacts (pins) of one or more terminal connectors. In one embodiment, two host contacts 15 are arranged on the first side surface 111 of the housing 11. Moreover, as compared with the two host contacts 15, the switch mechanism 13 is nearer to the top surface 113 of the housing 11; in other words, in this embodiment, a distance between the top surface 113 of the housing 11 and the switch mechanism 13 is less than a distance between the top surface 113 of the housing 11 and each of the two host contacts 15. Furthermore, along a width direction of the first side surface 111, as compared with the host contact 15 at the rear side (the host contact 15 at the right portion of FIG. 2), the switch mechanism 13 is nearer to the host contact 15 at the front side (the host contact 15 at the left portion of FIG. 2); in other words, in this embodiment, along the width direction of the first side surface 111, a distance between the host contact 15 at the front side and the switch mechanism 13 is less than a distance between the host contact 15 at the rear side and the switch mechanism 13. Specifically, in one embodiment, the switch mechanism 13 may be arranged on a center C along the width direction of the first side surface 111, while the middle portion between the two host contacts 15 on the first side surface 111 is offset toward a direction away from the front side surface 114 from the center C along the width direction of the first side surface 111. The functions corresponding to such feature will be described in the embodiment shown in FIG. 14.

As shown in FIG. 3, in one embodiment, the two host contacts 15 are arranged on the second side surface 112 of the housing 11. In one embodiment, as shown in FIG. 2 and FIG. 3, the two host contacts 15 on the second side surface 112 and the two host contacts on the first side surface 111 may be arranged at left and right sides of the housing 11 symmetrically. Moreover, the rotation plate 17 is arranged on the top surface 113 of the housing 11, and the footpads 19 are arranged on a bottom surface 115 of the housing 11. The rotation plate 17 is configured to allow the user to adjust the current, the voltage, or the energy intensity output by the host 10. In one embodiment, the footpads 19 are configured to prevent the host 10 from being moved with respect to a plane (for example, a desk plane or a ground).

It is understood that, the number and the position of the switch mechanism 13 and the host contacts 15 of the host 10 can be adjusted according to user's demands and are not limited by the embodiments shown in FIG. 1 to FIG. 3. For example, in some embodiments, the two host contacts 15 on the second side surface 112 may be omitted, and the host contacts 15 are only arranged on the first side surface 111. For another example, the switch mechanism 13 originally on the first side surface 111 may be arranged on the second side surface 112 or may be arranged on both the first side surface 111 and the second side surface 112. In some exemplary embodiments, the switch mechanism 13 is neither arranged on the first side surface 111 nor the second side surface 112 of the housing 11; instead, the switch mechanism 13 is arranged on an object away from the host 10, and the switch mechanism 13 is connected to the host 10 through a signal cable.

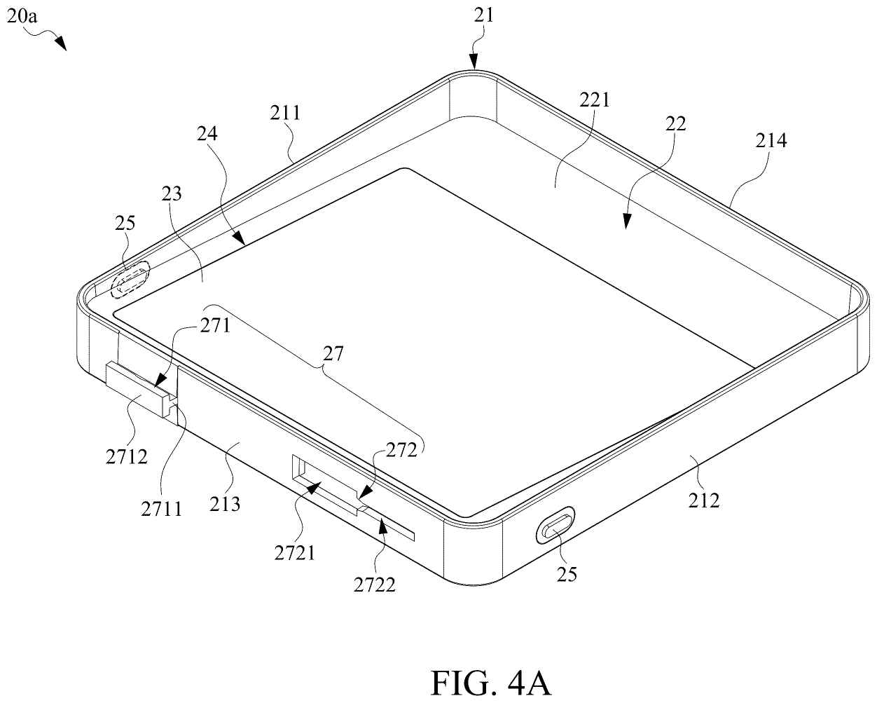

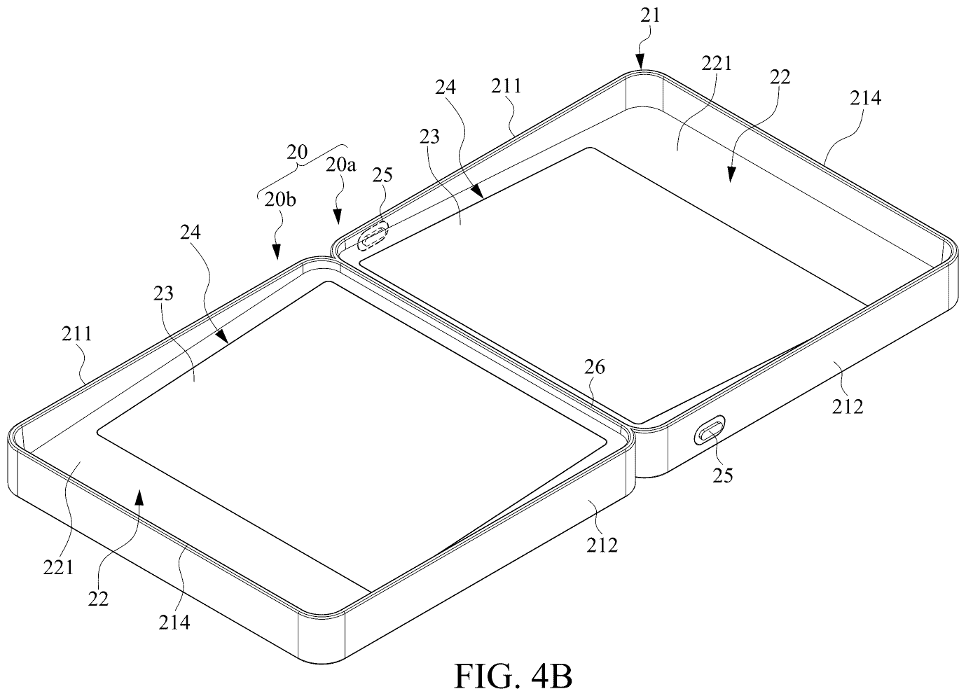

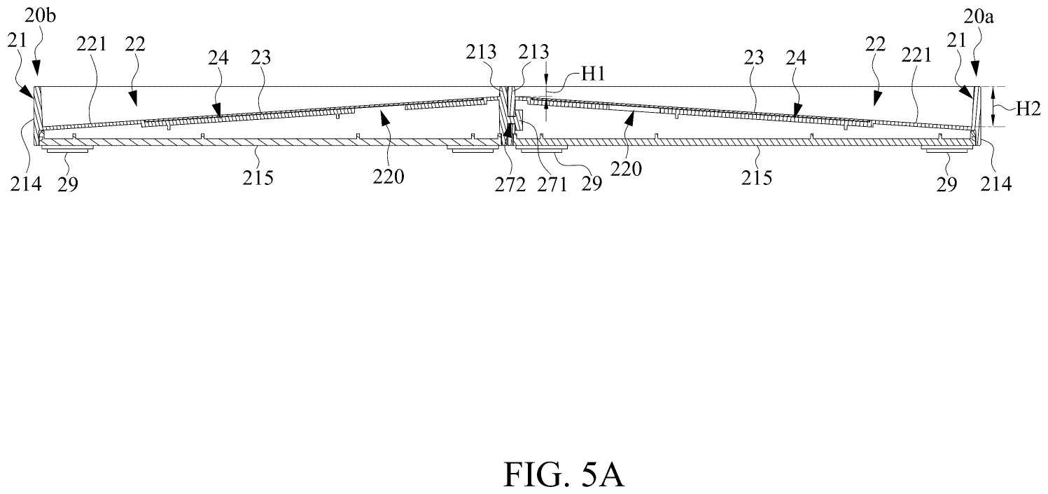

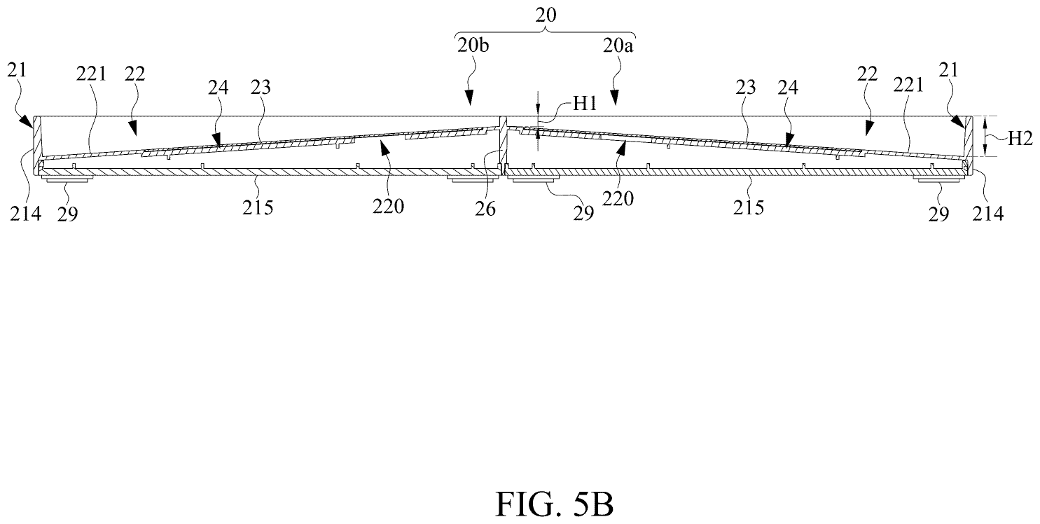

FIG. 4A illustrates a schematic view of an accommodation module 20a according to an embodiment of the instant disclosure. FIG. 5A illustrates a cross-sectional view of two accommodation modules 20a, 20b according to an embodiment of the instant disclosure, where the two accommodation modules 20a, 20b are assembled with each other. Please refer to FIG. 4A and FIG. 5A. In some embodiments, the accommodation module 20a comprises a tank body 21, an electrode 23, at least one tank contact 25, a connection structure 27 (as shown in FIG. 4A), and a plurality of footpads 29. The tank body 21 has a rectangular structure and comprises two side plates 211, 212, a front-end plate 213, a rear-end plate 214, and a bottom plate 215. The two side plates 211, 212 are at left and right sides of the tank body 21, the front-end plate 213 is at the front of the tank body 21 and connected between the two side plates 211, 212, and the rear-end plate 214 is opposite to the front-end plate 213 and connected between the two side plates 211, 212. A recess 22 is in the tank body 21, and a bottom surface 221 of the recess 22 comprises an inclination region. The footpads 29 are arranged below the bottom plate 215 to prevent the accommodation module 20a from being moved with respect to a plane (for example, a desk plane or a ground).

As shown in FIG. 5A, the bottom surface 22a of the recess 22 of the accommodation module 20a is inclined with respect to the bottom plate 215, where a height difference H1 is between one end of the bottom surface 221 adjacent to the front-end plate 213 and an upper edge of the front-end plate 213, a height difference H2 is between one end of bottom surface 221 adjacent to the rear-end plate 214 and an upper edge of the rear-end plate 214, and the height difference H2 is greater than the height difference H1. In some embodiments, the height difference H1 is less than the average size of a user's arch (where the user belongs to a certain group; the certain group may be defined by, e.g., race, region, gender, or age range); for example, the height difference H1 is less than 1.5 cm. It is understood that, in some embodiments, an excessive height difference H1 may make the user's arch uncomfortable when the user's feet step on the accommodation modules 20a, 20b; moreover, the user's feet may even break the front-end plate 213. In some embodiments, the height difference H1 is greater than 0 cm; the increase of the height difference H1 facilitates the amount of liquid capable of being accommodated in the recess 22 to be increased and prevents the liquid in the recess 22 from being spilled off, and the increase of the height difference H1 also prevents the liquid or the conductive medium accommodated in the adjacent two accommodation modules 20a, 20b from being in communication with each other which thus causes short-circuited condition. In some embodiments, the height difference H2 is less than the average thickness of a user's palm (where the user belongs to a certain group; the certain group may be defined by, e.g., race, region, gender, or age range); for example, the height difference H2 is less than 3 cm. It is understood that, in some embodiments, an excessive height difference H2 may make the user's wrist uncomfortable (the reason may lies in that the user's wrist is pressed against the rear-end plate 214 for a long time to cause discomfort or the user has to raise his/her wrist which thus cause his/her wrist to get sore easily) (the arrangement regarding how the user's two hands are placed on the apparatus can be referred to FIG. 14); moreover, the user's feet may collide the rear-end plate 214 easily and thus break the rear-end plate 214. On the other hand, an insufficient height difference H2 may cause the space of the recess 22 for accommodating the liquid or the conductive medium to be not enough. In some embodiments, the height difference H2 is less than a height of the switch mechanism 13 of the host 10 with respect to the bottom surface 115 of the housing 11.

It is understood that, although in the embodiments shown in FIG. 4A and FIG. 5A, the whole region of the bottom surface 221 of the recess 22 is inclined with respect to the bottom plate 215, the instant disclosure is not limited thereto. In other exemplary embodiments, a region of the bottom surface 221 of the recess 22 adjacent to the front-end plate 213 is inclined, while a region of the bottom surface 221 of the recess 22 adjacent to the rear-end plate 214 is flat. In some other exemplary embodiments, the bottom surface 221 of the recess 22 has several inclined regions with different inclined angles. According to some embodiments, by configuring the bottom surface 221 of the recess 22 to be inclined, the accommodation module can thus have a sufficient depth, thereby allowing the liquid or the wet medium (conductive medium) to cover the purlicue of the user' hand to achieve the therapy purpose. In the embodiments for foot therapy, the bottom surface 221 of the recess 22 with the inclined configuration allows the user's arch to properly attach to the bottom surface 221 of the recess 22, thereby improving user's comfort.

In one embodiment, the electrode 23 is arranged on the bottom surface 221 of the recess 22; for example, the electrode 23 is fixed on the bottom surface 221 of the recess 22. As shown in FIG. 5A, in one embodiment, the recess 22 further comprises a positioning structure 24 for positioning the electrode 23. In one embodiment, the positioning structure 24 is recessed from the bottom surface 221 of the recess 22, and the shape of the positioning structure 24 corresponds to the shape of the electrode 23. In one embodiment, the electrode 23 is arranged on the positioning structure 24 so that the upper surface of the electrode 23 is coplanar with the bottom surface 221, thereby preventing the user from being hurt by the edge of the electrode 23 or preventing the user from detaching the electrode 23 from the recess 22. In addition, in this embodiment, the lower portion of the positioning structure 24 further has a through hole 220 for the wires connecting to the electrode 23 to pass through. In some embodiments, the tank body 21 is of a sealed configuration to prevent water from leaking into the interior of the tank body 21 which will thus cause the short-circuited condition of the wires. For example, a waterproof strip (not shown) may be provided between the electrode 23 and the positioning structure 24, thereby preventing the liquid from entering the interior of the tank body 21 through the gap between the electrode 23 and the positioning structure 24. In some embodiments, the front-end plate 213 is nearer to the positioning structure 24 as compared with the rear-end plate 214, so that the electrode 23 is adjacent to the front-end plate 213 when the electrode 23 is arranged on the positioning structure 24. Furthermore, because the user's body portion is arranged across two accommodation modules (as shown in FIG. 14), by configuring the electrode 23 to be adjacent to the front-end plate 213, the portions of the user's body covered by the electrode 23 can be maintained at equipotential as possible as they can, thereby preventing the electrical field attenuation caused by the increase of distance and thus improving the therapeutic efficiency. For example, when the middle line of user's palm corresponds to the front-end plate 213, the electrode 23 can cover most parts of the user's hand (including the palm and fingers) to be treated.

In one embodiment, the two tank contacts 25 are symmetrically arranged at left and right sides of the tank body 21, and as compared with the rear-end plate 214, the front-end plate 213 is nearer to the two tank contacts 25; in other words, in this embodiment, a distance between the front-end plate 213 and the two tank contacts 25 is less than a distance between the rear-end plate 214 and the two tank contacts 25. The two tank contacts 25 are electrically connected to the electrode 23, so that the current can be transmitted from the tank contacts 25 to the electrode 23 or transmitted from the electrode 23 to the tank contacts 25. In some embodiments, the shapes of the two tank contacts 25 correspond to the shapes of the two host contacts 15. For example, as shown in FIG. 3 and FIG. 4A, the tank contact 25 is a male structure, and the host contact 15 is a female structure. When the tank contact 25 is connected to the host contact 15, the tank contact 25 is inserted into the host contact 15 so that the electrical connection between the tank contact 25 and the host contact 15 is achieved. In some embodiments, magnetic members are provided in the two host contacts 15 and the two tank contacts 25, so that the two host contacts 15 and the two tank contacts 25 are configured as magnetic terminal connectors. Accordingly, in these embodiments, the host contacts 15 and the tank contacts 25 are connected to each other through magnetic forces, and thus the accommodation module 20a can be prevented from being detached off the host 10. In the embodiment that both the host contacts 15 and the tank contacts 25 are magnetic terminal connectors, the footpads 19 below the host 10 or the footpads 29 below the accommodation module 20a are devoid of the skid proof function. Therefore, by using the magnetic force between the host contact 15 and the tank contact 25, the host 10 and the accommodation module 20a can be moved toward each other through sliding and thus connected to each other. For example, in one embodiment, the footpads 19 of the host 10 do not have the skid proof function, while the footpads 29 of the accommodation module 20a have the skid proof function. Alternatively, in another embodiment, the footpads 19 of the host 10 have the skid proof function, while the footpads 29 of the accommodation module 20a do not have the skid proof function.

In some embodiments, except the wires connecting the tank contacts 25 with the electrode 23, the accommodation module 20a does not have additional circuits or electronic components. By collectively configuring the components for current control inside the host 10, the manufacturing cost of the accommodation module 20a can be reduced, and the replacement of an accommodation module 20a which is to be replaced by a new accommodation module 20a can be achieved conveniently. Furthermore, in some embodiments, because complicated circuit components (e.g. current controller or voltage booster) are no more necessarily required to be arranged in the accommodation module 20a, the height of the accommodation module 20a can be lowered. Therefore, when the therapy is performed on the user, the user' hand can be placed on the desk properly, thereby improving the user experience. In some embodiments, by integrating the electrode 23 and the wires connecting to the tank contact 25 and the electrode 23 with the accommodation module 20a, several advantages can be provided. For instance, with the integrated configuration mentioned above, the user does not need to connect the electrode 23 to additional adapter wires (for example, in an apparatus known to the inventor, the user has to respectively connect a plurality of adapter wires to a plurality of electrodes 23 and then connect the adapter wires to the host 10) upon operating the apparatus. Through such configuration, the assembling complexity of the apparatus can be lowered, thereby increasing the user experience. Moreover, when the electrode 23 is oxidized, the user can replace the whole accommodation module 20a, and the user does not need to disassemble the electrode 23 from the tank body 21 which may cause the damage of the apparatus and thus affecting the product safety or efficiency. In one embodiment, a sufficient space is provided below the inclined bottom surface 221 (for example, the space may be provided below the region of the bottom surface 221 adjacent to the front-end plate 213 (as shown in FIG. 5A)), so that the tank contacts 25 and the wires can be arranged in the space and the tank contacts 25 can be electrically connected to the electrode 23.

The connection structure 27 is configured to allow two adjacent accommodation modules to be detachably connected to each other. In some embodiments, the connection structure 27 is arranged on the front-end plate 213 of the accommodation module 20a and comprises a protrusion 271 and an opening 272. The protrusion 271 has a T-shaped cross-section and comprises a neck portion 2711 and a head portion 2712. The neck portion 2711 is fixed on the outer surface of the front-end plate 213, and the head portion 2712 is connected to an end portion of the neck portion 2711. Along a vertical direction, the width of the neck portion 2711 is less than the width of the head portion 2712. The opening 272 is arranged adjacent to the protrusion 271 and comprises a containing portion 2721 and a fixation portion 2722. The containing portion 2721 is arranged adjacent to the protrusion 271, and the fixation portion 2722 is at the outer side of the containing portion 2721. Along the vertical direction, the width of the fixation portion 2722 is less than the width of the containing portion 2721, and the width of the fixation portion 2722 is slightly greater than the width of the neck portion 2711. In some embodiments, the connection structures 27 of the accommodation modules 20a, 20b may be replaced by a pivotally rotation mechanism (not shown). In this embodiment, the pivotally rotation mechanism is connected between the front-end plates 213 of the two accommodation modules, and the two accommodation modules which are connected to each other can be operated between a closed state (where the recesses 22 or the bottom plates 215 of the two accommodation modules are opposite to each other) and an opened state (where the two accommodation modules are side-by-side placed) through the pivotally rotation mechanism. In some other embodiments, the accommodation modules 20a, 20b may be connected to each other by using magnetic attraction or other manners. For example, the front-end plates 213 of the two accommodation modules may be connected to each other through the magnetic forces provided by the paired magnetic members arranged on the back surfaces of the front-end plates 213 of the two accommodation modules.

In some other embodiments, as shown in FIG. 4B and FIG. 5B, the front-end plate 213 of the two accommodation modules is a shared plate and defined as an intermediate plate 26, the intermediate plate 26 is arranged and connected between the side plates 211, 212. In this embodiment, the accommodation modules 20a, 20b are connected to each other through the intermediate plate 26 to form accommodation tanks 20; for example, the accommodation modules 20a, 20b and the intermediated 26 may be integrally formed as a one-piece structure. In some embodiments, the side plates 211 (or the side plates 212) of the accommodation modules 20a, 20b may be connected to each other to form the side plate of the accommodation tank 20; in some other embodiments, the side plates 211 (or the side plates 212) of the accommodation modules 20a, 20b may be respectively arranged on the accommodation modules 20a, 20b and together form the side plate of the accommodation tank 20. In some embodiments, the intermediate plate 26 may be only arranged on the bottom surface 221 of the recess 22 (the configuration is not shown); in these embodiments, the recesses 22 of the accommodation modules 20a, 20b are separated by the intermediate plate 26, and the recesses 22 of the accommodation modules 20a, 20b are in communication with each other through the accommodation spaces below the bottom surfaces 221 of the accommodation modules 20a, 20b. In some other embodiments, the intermediate plate 26 extends upward from the bottom surface 221 of the recess 22 toward the bottom plate 215 (as shown in FIG. 5B), and the accommodation spaces below the bottom surfaces 221 of the accommodation modules 20a, 20b are separated by the intermediate plates 26. The wires electrically connected between the electrode 23 and the tank contact 25 can be arranged in the accommodation space. In some embodiments, the wires of the accommodation module 20a pass through the intermediate plate 26 (not shown) and are electrically connected to the electrode 23 (or the tank contact 25) of the accommodation module 20b. The tank contacts 25 may be arranged on the side plate 211 (or the side plate 212) of the accommodation module 20a (or the accommodation module 20b), and the tank contacts 25 are electrically connected to the electrodes 23 of the accommodation modules 20a, 20b, respectively. In this embodiment, a host contact 15 with multiple pins may be arranged on the first side surface 111 of the host 10 so as to be electrically connected to the tank contacts 25. As for the accommodation tank 20 in this embodiment, detailed configurations of the structural features of the accommodation modules 20a, 20b may be referred to the structural features of the accommodation module of other embodiments.

In some embodiments, the structures of the accommodation modules 20b, 20c, 20d are identical to the structure of the accommodation module 20a. For the purpose of simplification, the structural features of the accommodation modules 20b, 20c, 20d will not be repeated, and in the following paragraphs, the components of the accommodation modules 20b, 20c, 20d will be given the same reference numerals as the components of the accommodation module 20a. According to some embodiments, by adopting the accommodation modules 20a, 20b, 20c, 20d with identical configurations, the manufacturing costs for the apparatus can be lowered and the assembling complexity of the apparatus can be reduced. Moreover, when the electrodes 23 of the accommodation modules 20a, 20b, 20c, 20d are oxidized so that the conductivity of the electrodes 23 decreases, the accommodation modules with oxidized electrodes 23 can be replaced by new accommodation modules with identical specifications. However, it is understood that, the appearances of the accommodation modules 20a, 20b, 20c, 20d can be properly adjusted according to different demands.

According to one embodiment of the instant disclosure, the method for assembling the iontophoresis apparatus 1 is provided in the following paragraphs.

The method for assembling the iontophoresis apparatus 1 comprises respectively connecting the two accommodation modules 20a, 20b with each other and respectively connecting the two accommodation modules 20c, 20d with each other. For example, as shown in FIG. 5A, upon connecting the accommodation modules 20a, 20b with each other, firstly the front-end plates 213 of the accommodation modules 20a, 20b are opposite to each other, and the protrusion 271 of one of the two accommodation modules 20a, 20b is inserted into the containing portion 2721 of the opening 272 of the other one of the two accommodation modules 20a, 20b (as shown in FIG. 4A). After the protrusion 271 is inserted into the containing portion 2721, the two accommodation modules 20a, 20b are laterally moved with respect to each other, so that the neck portion 2711 of the protrusion 271 (as shown in FIG. 4A) enters the fixation portion 2722 of the opening 272 (as shown in FIG. 4A). Because the width of the fixation portion 2722 (as shown in FIG. 4A) along the vertical direction is slightly greater than or equal to the width of the neck portion 2711 (as shown in FIG. 4A), after the neck portion 2711 of the protrusion 271 (as shown in FIG. 4) is moved into the fixation portion 2722 of the opening 272 (as shown in FIG. 4A), the neck portion 2711 can be firmly mated in the fixation portion 2722 (as shown in FIG. 4A). Accordingly, the connection between the two accommodation modules 20a, 20b can be achieved. Likewise, the accommodation modules 20c, 20d can be connected to each other similarly.

The method for assembling the iontophoresis apparatus 1 further comprises connecting the accommodation modules 20a, 20b, 20c, 20d to the host 10. For example, as shown in FIG. 6, the accommodation modules 20a, 20b are coupled to the host 10 through the electrical connection between the two tank contacts 25 of the accommodation modules 20a, 20b and the two host contacts 15 on the first side surface 111 of the host 10, and the accommodation modules 20c, 20d are coupled to the host 10 through the electrical connection between the two tank contacts 25 of the accommodation modules 20c, 20d and the two host contacts 15 on the second side surface 112 of the host 10. After the accommodation modules 20a, 20b, 20c, 20d are coupled to the host 10, the iontophoresis apparatus 1 is in an expanded state, and the user can place the conductive medium and liquid in the accommodation modules 20a, 20b, 20c, 20d to prepare performing the iontophoresis therapy.

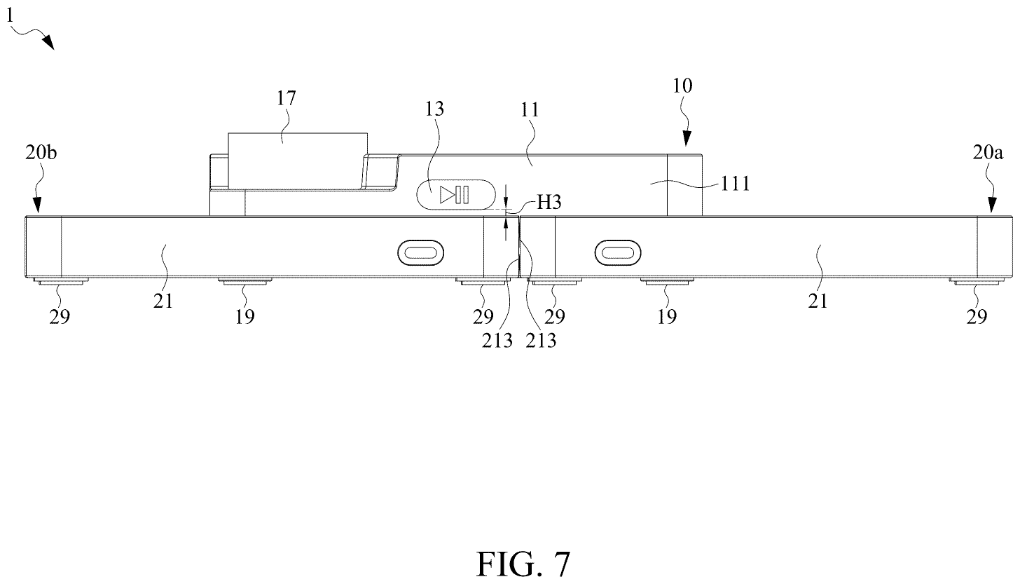

It is understood that, when the iontophoresis apparatus 1 is in the expanded state, the switch mechanism 13 on the side surface of the host 10 is not shielded by any of the accommodation modules 20a, 20b, 20c, 20d, so that the user can press the switch mechanism 13 conveniently. For example, as shown in FIG. 7, when the iontophoresis apparatus 1 is in the expanded state, the switch mechanism 13 is higher than the tan bodies 21 of the two accommodation modules 20a, 20b. A height difference H3 is between the switch mechanism 13 and the upper edge of the side plate of the tank body 21 (for example, the side plates 211, 212 shown in FIG. 4A). The height difference H3 is designed so that the user's finger can press the switch mechanism 13 conveniently. For example, the height difference H3 is less than the average thickness of the finger portion of a user (which is about 2 cm). Moreover, the configuration of the height difference H3 also prevents the liquid from the accommodation modules 20a, 20b, 20c, 20d from passing through the gaps of the switch mechanism 13 and thus enter the interior of the host 10.

On the other hand, although in the embodiment shown in FIG. 6, the accommodation modules 20a, 20b, 20c, 20d are connected to the host 10 at the same time, the instant disclosure is not limited thereto. According to user demands, the user can connect the accommodation module on one side of the host 10. For example, the user can merely connect the accommodation modules 20a, 20b on the first side surface 111 of the host 10 or merely connect the accommodation modules 20c, 20d on the second side surface 112 of the host 10. Accordingly, the user can choose just performing a one-hand/foot therapy, and the user can do other activities with the other hand (for example, operating the mouse or the mobile phone); on the other hand, the user can perform therapy on two hands/feet at the same time to reduce the total therapy duration.

Please continue to refer to FIG. 6. In one embodiment, the housing 11 has a stepped structure 12 formed on the top surface 113 and is at a corner intersected by the first side surface 111 and the front side surface 114. The stepped structure 12 comprises a standing surface 121 and a bottom surface 125. The standing surface 121 is between the top surface 113 and the bottom surface 125 and perpendicular to the top surface 113 and the bottom surface 125. In this embodiment, the standing surface 121 is formed by a first plane 122, a curved surface 123, and a second plane 124. The first plane 122 is connected to the front side surface 114 and inclined with respect to the front side surface 114, the second plane 124 is connected to the first side surface 111 and inclined with respect to the first side surface 111, and the curved surface 123 is connected between the first plane 122 and the second plane 124. The inclination angle of the first plane 122 with respect to the front side surface 114 may be the same as the inclination angle of the second plane 124 with respect to the front side surface 114, for example, the inclination angle may be 45 degrees.

In some embodiments, the rotation plate 17 passes through the bottom surface 125 of the stepped structure 12, and at least a portion of the peripheral surface of the rotation plate 17 is covered by the curved surface 123. Through the configuration, when the user rotates the rotation plate 17, the rotation angle of the user's hand will be limited by the first plane 122 and the second plane 124, thereby preventing the apparatus from outputting excessive current caused by overly tuning the rotation plate 17. In an exemplary embodiment, the value of the current for performing therapy on the foot is twice the value of the current for performing therapy on the hand. Furthermore, the rotation interval of the rotation plate 17 that is within a single rotation performed by the user at most allows the value of the current to be adjusted to twice the value of the current for performing therapy on the hand. Accordingly, in this embodiment, if the therapy is performed on the foot, the user has to rotate the rotation plate 17 twice to allow the host 10 to output the value of the current for performing therapy on the foot.

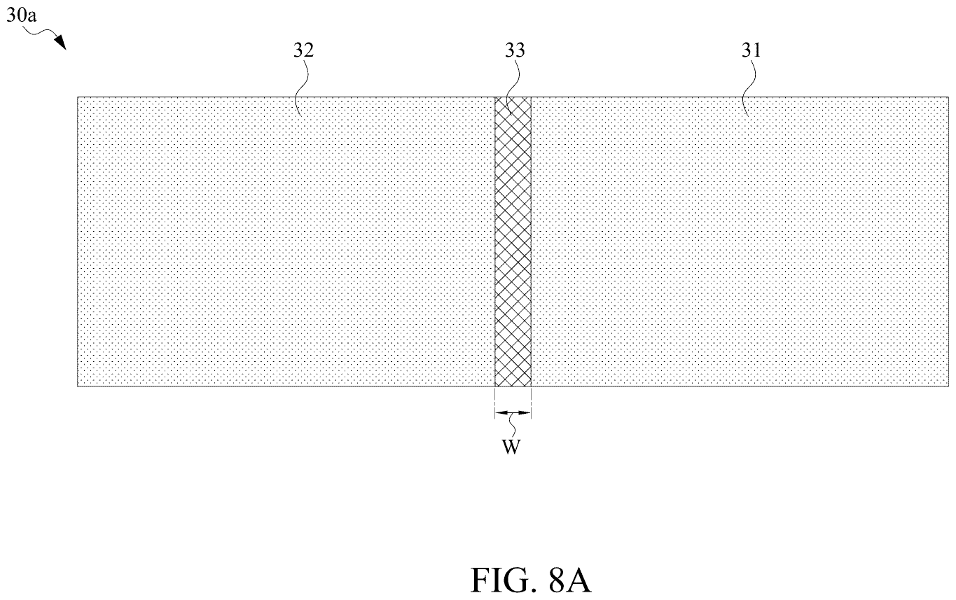

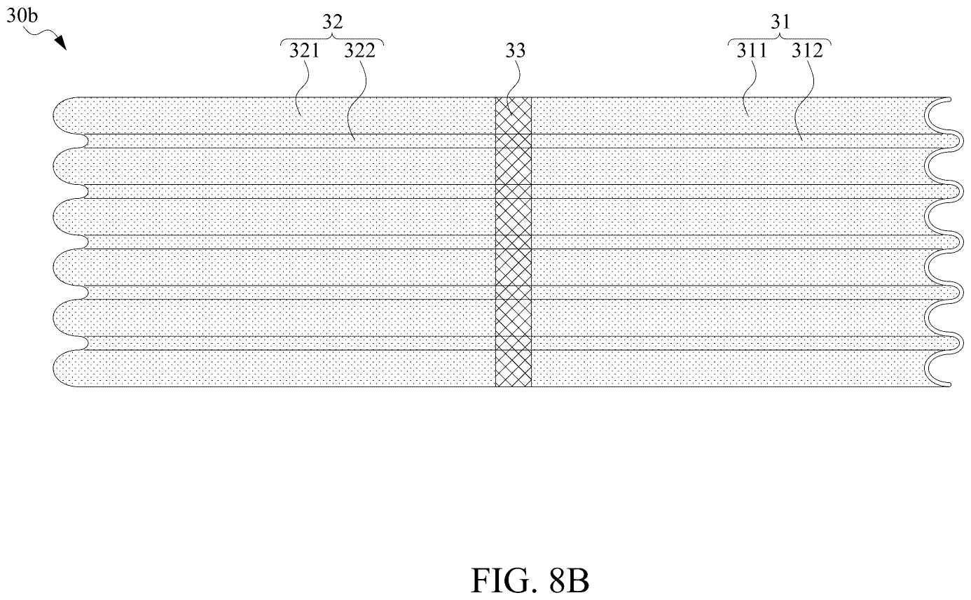

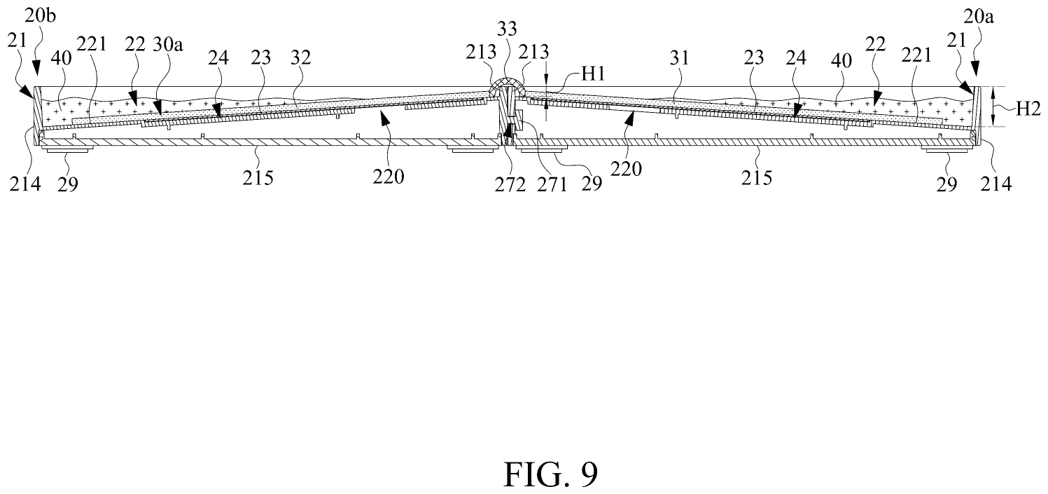

FIG. 8A illustrates a schematic view of a conductive medium according to an embodiment of the instant disclosure. FIG. 9 illustrates a cross-sectional view of two accommodation modules 20a, 20b according to an embodiment of the instant disclosure. The conductive medium comprises a material capable of absorbing liquid, for example, a fabric, a foam, or a gel. In some embodiments, the conductive medium is a fabric 30a, where the fabric 30a may be arranged on the two accommodation modules 20a, 20b and the liquid 40 (for example, water or beauty care liquid) may be filled in the two accommodation modules 20a, 20b. In one embodiment, as shown in FIG. 8A, the fabric 30a comprises a first absorbent section 31, a nonabsorbent section 33 (a water-repellant section), and a second absorbent section 32. The first absorbent section 31 and the second absorbent section 32 may be made by fabrics which can absorb liquid easily or made by non-fabrics which can absorb liquid. On the other hand, the nonabsorbent section 33 may be made by fabrics or non-fabrics which cannot absorb liquid (water repellent); moreover, the fabrics or non-fabrics which cannot absorb liquid (water repellent) may be electrically insulated. The width W of the nonabsorbent section 33 may be twice the height difference H1, so that the area of the fabric 30a which cannot provide the therapy function can be reduced. In some embodiments, the bottom surface of the fabric 30a is made of a nonabsorbent material, two absorbent sheets which can absorb liquid easily are fixed (for example, through sewing, laminating, or gluing) on the fabric 30a to form the first absorbent section 31 and the second absorbent section 32, and a width W is maintained between the two absorbent sheets so that the nonabsorbent material at the bottom surface of the fabric 30a can be exposed to form the nonabsorbent section 33. In other some embodiments, the two absorbent sheets are respectively fixed at left and right sides of a nonabsorbent sheet. In some embodiments, to prevent the first absorbent section 31 and the second absorbent section 32 from being in contact with each other, an upper surface of one of the two absorbent sheets is fixed on a lower surface of the nonabsorbent sheet, and a lower surface of the other one of the two absorbent sheets is fixed on an upper surface of the nonabsorbent sheet, so that the boundaries of the first absorbent section 31 and the second absorbent section 32 can be separated by the nonabsorbent section 33.

FIG. 8B illustrates a schematic view of a conductive medium according to another embodiment of the instant disclosure. As shown in FIG. 8B, in some embodiments, the surfaces of the first absorbent section 31 and the second absorbent section 32 may have protruding structures (for example, fuzzes, wrinkles, or cloth patches), so that every portion of the user's hand/foot (for example, side portions of the fingers, side portions of the palm, the purlicue, or the side portions of the foot of the user) can be contacted by the conductive medium or the user can hold the protruding structures with fingers. For example, the first absorbent section 31 and the second absorbent section 32 of the fabric 30b shown in FIG. 8B have wrinkles which respectively have a plurality of protruding portions 311, 321 and a plurality of recessed portions 312, 322. The user can place the fingers in the recessed portions 312, 322 so that the protruding portions 311, 321 are between the fingers to perform the therapy on the side portions of the user' fingers. In some embodiments, after an absorbent sheet is folded to form wrinkles, a nonabsorbent sheet is sewed on the boundary of the absorbent sheet to position the wrinkles, thereby forming the fabric 30b. Alternatively, in some other embodiments, the boundaries between the protruding portions 311, 321 and the recessed portions 312, 322 (along the left-right direction shown in FIG. 8B) are sewed to position the wrinkles. Alternatively, in some other embodiments, after the fabric 30a is folded to form wrinkles, the fabric 30a is then sewed along the left boundary of the second absorbent section 32 and the right boundary of the first absorbent section 31 (the upper-lower direction shown in FIG. 8B) to position the wrinkles, thereby forming the fabric 30b. In some embodiments, the surface of the nonabsorbent section 33 may be provided with protruding structures.

As shown in FIG. 9, when the iontophoresis apparatus 1 is in the expanded state, the fabric 30a (or the fabric 30b) is arranged across the tank bodies 21 of the two accommodation modules 20a, 20b, where the first absorbent section 31 and the second absorbent section 32 respectively cover the two electrodes 23 in the two tank bodies 21, and the nonabsorbent section 33 extends along the two front-end plates 213 of the two tank bodies 21. According to some embodiments, through the configuration of the first absorbent section 31 and the second absorbent section 32 of the fabric 30a, the user's body portion can be immersed in the liquid while does not directly contact the electrode 23.

Moreover, through the configuration of the nonabsorbent section 33 of the fabric 30a, the first absorbent section 31 and the second absorbent section 32 are not in contact with each other to prevent short-circuited condition, thereby ensuring that the current can pass through the user's body portion. Therefore, compared with the embodiment where two separated absorbent fabrics are respectively arranged in the tank bodies 21 of the two accommodation modules 20a, 20b, in this embodiment, the user can arrange the fabric 30a on the two accommodation modules 20a, 20b quickly, thereby reducing the preparation time for performing the therapy. Moreover, the whole fabric 30a can be cleaned and dried in a one-time operation, respectively. In some embodiments, the thickness of the fabric 30a is less than the height difference H3 (as shown in FIG. 7), so that the user's body portion can contact the switch mechanism 13 when the user's body portion is placed on the fabric 30a. In some embodiments, the thickness of the fabric 30a is greater than half of the average thickness of the user's finger or palm (which may be in the range between 0.5 cm and 1 cm) to cover the side portions of the user's fingers or the purlicue of the user' hand.

FIG. 10 illustrates a schematic view of an iontophoresis apparatus 1 according to an embodiment of the instant disclosure, where the iontophoresis apparatus 1 is in a stored state. In some embodiments, the length and width of the accommodation modules 20a, 20b, 20c, 20d are respectively identical to the length and the width of the host 10. Therefore, after the accommodation modules 20a, 20b, 20c, 20d are detached from the host 10, the accommodation modules 20a, 20b, 20c, 20d can be stacked below the host 10. Hence, the iontophoresis apparatus 1 can be stored without occupying a larger space, such that the iontophoresis apparatus 1 can be stored and carried conveniently.

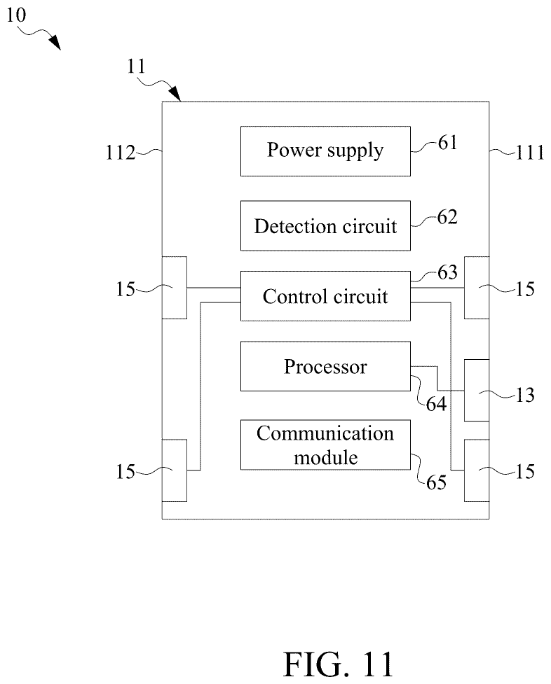

FIG. 11 illustrates a block diagram of a host 10 according to an embodiment of the instant disclosure. In one embodiment, the host 10 further comprises a power supply 61, a detection circuit 62, a control circuit 63, a processor 64, and a communication module 65. The power supply 61 is configured to supply electricity for the host 10. The detection circuit 62 is configured to determine a connection state between the accommodation modules 20a, 20b, 20c, 20d and the host 10 or determine whether the control circuit 63 functions normally or not. The control circuit 63 is configured to control the current output of the host contact 15. The processor 64 is configured to process and control a control signal and/or a detection signal of the host 10. For example, the processor 64 is configured to operate the detection circuit 62 and the control circuit 63. Alternatively, in some embodiments, the processor 64 is configured to receive a driving signal from the switch mechanism 13 and adjust the current supplying to the control circuit 63 according to the driving signal. The communication module 65 is configured to allow the host 10 to transmit information to a user's terminal device through wireless signals or to receive the control command transmitted from the user's terminal device, so that a remote intelligent medical service purpose according to some embodiments can be achieved through the IoT architecture. Moreover, the communication module 65 is also configured to allow the terminal device to perform data collection on the host 10 for tracking usage status or setting parameters, so that the machine learning database for user therapy records can be established. It is understood that, although in the embodiment shown in FIG. 11, the processor 64 and the communication module 65 are arranged separately, the instant disclosure is not limited thereto. In some other embodiments, the processor 64 and the communication module 65 may be integrated into a single component. Moreover, the processor 64 may be formed by two or more components; for example, the processor 64 comprises a processing chip and a microcontroller chip.

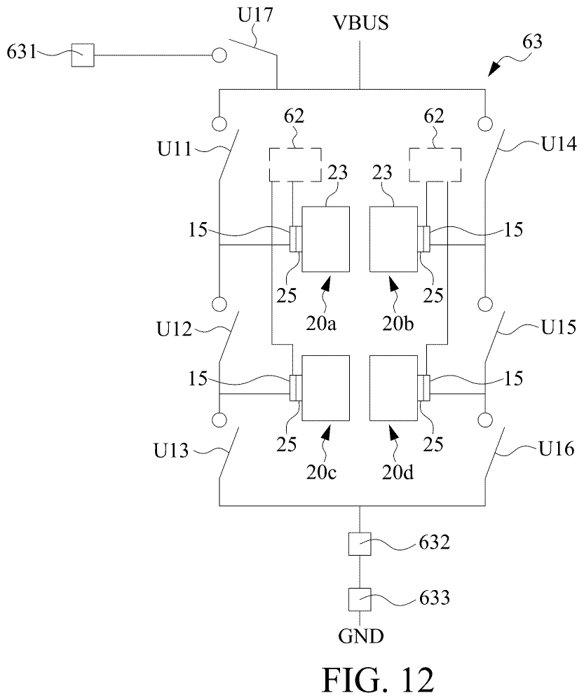

FIG. 12 illustrates a circuit diagram of a control circuit 63 and a detection circuit 62 of a host 10 according to an embodiment of the instant disclosure. In some embodiments, the control circuit 63 comprises a plurality of switches (for example, switches U11, U12, U13, U14, U15, U16, U17, where the switches U11, U12, U13, U17 are respectively referred to as the first switch U11, the second switch U12, the third switch U13, and the test circuit switch U17), a test signal source 631, a constant current source element 632, a current measurement element 633, and a voltage source VBUS. The switches U11, U12, U13, U14, U15, U16 are formed as an improved bridge circuit, where the switches U11, U12, U13 are cascaded with each other, and the switches U14, U15, U16 are cascaded with each other. According to some embodiments, by controlling the configuration of the switches U11, U12, U13, U14, U15, U16, the host contact 15 for which the host 10 transmits through can be adjusted. Accordingly, in some embodiments, the host 10 can output the current just through the host contacts 15 at one side of the host 10, or the host 10 can alternately output the currents with identical values through the host contacts 15 at different sides, or the host 10 can change the current transmission direction. The switches each may be an electrical switch, for example, a relay, an optical coupled switch, a field effect transistor, a bipolar transistor, or an insulated gate bipolar transistor.

The test signal source 631 is connected to the host contact 15 through the switch U17 and the switches U11, U12, U13, U14, U15, U16. In some embodiments, the host contact 15 is a terminal connector and comprises two pins, where one of the two pins is adapted to be electrically connected to the test signal source 631, and the other one of the two pins is electrically connected to the detection circuit 62. Likewise, the tank contact 25 is a terminal connector 25 and comprises two pins, and the two pins are short-circuited (for example, the two pins are both electrically connected to the electrode 23). Therefore, according to some embodiments, by controlling of the switches U11, U12, U13, U14, U15, U16, U17 to be turned on or off, the signal transmitted from the test signal source 631 can be transmitted to the tank contact 25 through one of the pins of the chosen host contact 15, and the signal can then be transmitted to the detection circuit 62 through the tank contact 25 and the other one of the pins of the chosen host contact 15. In one embodiment, the detection circuit 62 may comprise a comparator electrically connected to the processor 64, and the comparator is switched to a high potential or a low potential after the comparator receives the signal.

In some embodiments, the test signal transmitted from the test signal source 631 (for example, 5V potential) is adapted to execute a connection state determination operation. According to some embodiments, the purpose of the connection state determination operation is to determine the connection state between the accommodation modules 20a, 20b, 20c, 20d and the host 10. Under the test mode, the processor 64 controls the switch U17 to be turned on so as to output the test signal to the improved bridge circuit, and the control circuit 63 controls the switches of the improved bridge circuit to be sequentially turned on or off so as to control the output of the test signal. For example, in response to that the switch U11 is turned on and the switches U12, U13, U14, U15, U16 are turned off, the test signal then can be output to the accommodation module 20a. In another example, in response to that the switch U14 is turned on and the switches U11, U12, U13, U15, U16 are turned off, the test signal can be output to the accommodation module 20b. In another example, in response to that the switches U11, U12 are turned on and the switches U13, U14, U15, U16 are turned off, the test signal can be output to the accommodation module 20c. In another example, in response to that the switches U14, U15 are turned on and the switches U11, U12, U13, U16 are turned off, the test signal can be output to the accommodation module 20d. In response to that the detection circuit 62 detects that both the electrodes 23 connecting to the accommodation modules 20a, 20b transmit the test signal back to the detection circuit 62 but does not detect that both the electrodes 23 connecting to the accommodation modules 20c, 20d transmit the test signal back to the detection circuit 62, the detection circuit 62 determines that the first side surface 111 of the host 10 is connected to the accommodation modules 20a, 20b and the second side surface 112 of the host 10 is not connected to the accommodation modules 20c, 20d (or the detection circuit 62 determines that the accommodation modules 20c, 20d are not connected to the host 10 correctly; for example, when the detection circuit 62 only detects the test signal transmitted back to the detection circuit 62 from the accommodation module 20c, such condition indicates that the accommodation module 20d is not assembled correctly). In some other embodiments, the test signal transmitted from the test signal source 631 is adapted to execute an error determination operation. According to one or some embodiments, the purpose of the error determination operation is to detect whether the switches U11, U12, U13, U14, U15, U16 function normally. For example, to test whether the switch U11 functions normally, firstly the switches U17, U12, U13 are turned on and the switches U14, U15, U16 are turned off; in response to that the switch U11 is switched and the current measurement element 633 also detects that the signal is changed, the error determination operation determines that the switch U11 functions normally.

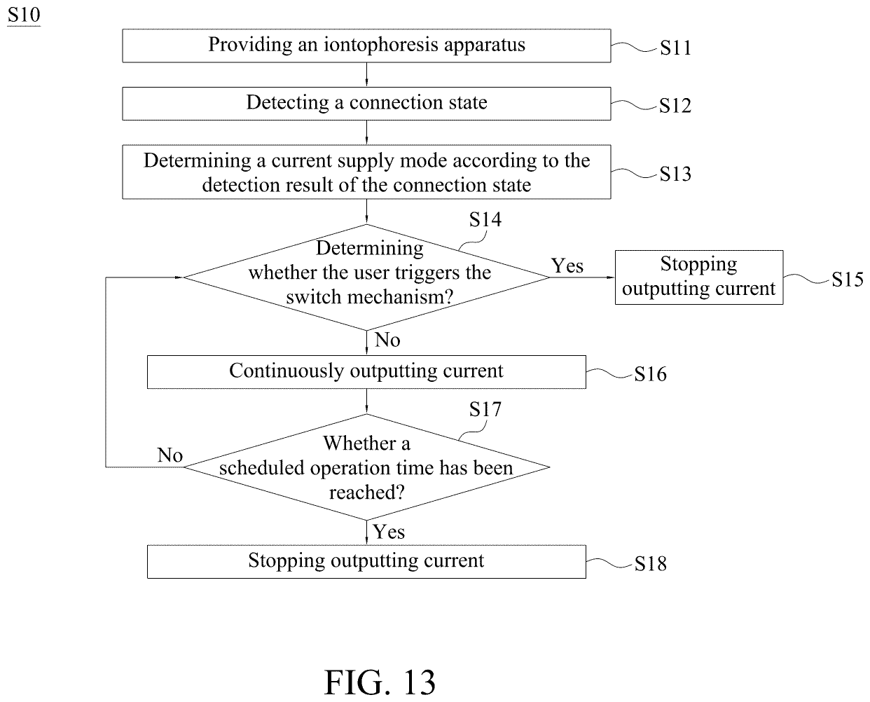

FIG. 13 illustrates a flowchart of a method for executing iontophoresis S10 according to an embodiment of the instant disclosure. For the sake of clarity, FIG. 13 will be described along with FIG. 1 to FIG. 12. Moreover, in different embodiments, some of the steps may be replaced or omitted.

In the step S11, an iontophoresis apparatus 1 according to any one of the foregoing embodiments is provided, and the accommodation modules 20a, 20b and/or the accommodation modules 20c, 20d of the iontophoresis apparatus 1 are at least connected to the host contacts 15 on the first side surface 111 or the host contacts 15 on the second side surface 112 of the host 10.

In the step S12, the processor 64 detects a connection state between the accommodation modules 20a, 20b, 20c, 20d and the host 10 through the test signal (for example, 5V potential) transmitted from the test signal source 631. The detection may be executed after the host 10 is booted, and the processor 64 can transmit an indication signal (for example, flashlight, sound) after the detection is completed to show the detection result. Specifically, according to some embodiments, please refer to FIG. 11 and FIG. 12, the processor 64 controls the switch U17 to be turned on to output the test signal transmitted from the test signal source 631 and instructs the control circuit 63 to adjust the test signal to be output to one of the host contacts 15. For example, the processor 64 firstly transmits a first test signal to the host contact 15 corresponding to the accommodation module 20a, and then the processor 64 transmits a second test signal to the host contact 15 corresponding to the accommodation module 20b. In response to that the processor 64 determines that the detection circuit 62 receives the first test signal and the second test signal, the processor 64 determines that the accommodation modules 20a, 20b are already connected to the host 10. In response to that the detection circuit 62 does not detect any of the first test signal and the second test signal, the processor 64 determines that accommodation modules 20a, 20b are not connected to the host 10. In some embodiments, after the processor 64 completes the detection of the connection state between the accommodation modules 20a, 20b, 20c, 20d and the host 10, the processor 64 controls the switch U17 to be turned off and controls the voltage source VBUS to be turned on.

In the step S13, according to the detection result of the step S12, a current supply mode is determined to output the current (the therapy mode is entered). Please refer to FIG. 6, FIG. 12, and FIG. 14. In some embodiments, in response to that the processor 64 determines that the accommodation modules 20a, 20b are connected to the first side surface 111 of the host 10 (as shown in FIG. 1) while the accommodation modules 20c, 20d are not connected to the second side surface 112 of the host 10, the processor 64 determines the condition as a one-side therapy mode. Under this configuration, the control circuit 63 is configured as a first mode: U11 (turned on), U12 (turned off), U13 (turned off), U14 (turned off), U15 (turned on), U16 (turned on), and the current sequentially passes through the electrode 23 of the accommodation module 20a, the user's body portion (for example, the body portion 71 shown in FIG. 14), and the electrode 23 of the accommodation module 20b and then back to the host 10. Alternatively, in some embodiments, the control circuit 63 is configured as a second mode: U11 (turned off), U12 (turned on), U13 (turned on), U14 (turned on), U15 (turned off), U16 (turned off), and the current sequentially passes through the electrode 23 of the accommodation module 20b, the user's body portion (for example, the body portion 71 shown in FIG. 14), and the electrode 23 of the accommodation module 20a and then back to the host 10. In some embodiments, the first mode and the second mode are alternately configured to prevent the risks of electric shock or chemical burning caused by charge accumulation. Moreover, in the first mode and the second mode, the host contacts 15 which are not connected to the accommodation modules 20c, 20d do not output current, thereby preventing the user from suffering electric shock upon the user unintentionally contacts the host contacts 15 which are not connected to the accommodation modules 20c, 20d.

Likewise, in response to that the processor 64 determines that the accommodation modules 20a, 20b are connected to the first side surface 111 of the host 10 (as shown in FIG. 1) while the accommodation modules 20c, 20d are not connected to the second side surface 112 of the host 10, the processor 64 also determines the condition as the one-side therapy mode. Under this configuration, the control circuit 63 is configured as a third mode: U11 (turned on), U12 (turned on), U13 (turned off), U14 (turned off), U15 (turned off), U16 (turned on), and the current sequentially passes through the electrode 23 of the accommodation module 20c, the user's body portion (for example, the body portion 72 shown in FIG. 14), and the electrode 23 of the accommodation module 20d and then back to the host 10. Alternatively, in some embodiments, the control circuit 63 is configured as a fourth mode: U11 (turned off), U12 (turned off), U13 (turned on), U14 (turned on), U15 (turned on), U16 (turned off), and the current sequentially passes through the electrode 23 of the accommodation module 20d, the user's body portion (for example, the body portion 72 shown in FIG. 14), and the electrode 23 of the accommodation module 20c and then back to the host 10. In some embodiments, the third mode and the fourth mode are alternately configured to prevent the risks of electric shock or chemical burning caused by charge accumulation. Moreover, in the third mode and the fourth mode, the host contacts 15 which are not connected to the accommodation modules 20a, 20b do not output current, thereby preventing the user from suffering electric shock upon the user unintentionally contacts the host contacts 15 which are not connected to the accommodation modules 20a, 20b.

On the other hand, in response to that the processor 64 determines that both the first side surface 111 and the second side surface 112 of the host 10 are connected to the accommodation modules 20a, 20b, 20c, 20d, the processor 64 determines the condition as a dual-side therapy mode. Under this configuration, the control circuit 63 is configured as a fifth mode: U11 (turned on), U12 (turned off), U13 (turned on), U14 (turned off), U15 (turned on), U16 (turned off), and the current sequentially passes through the electrode 23 of the accommodation module 20a, the right part of the user's body portion (for example, the body portion 71 shown in FIG. 14), the electrode 23 of the accommodation module 20b, the electrode 23 of the accommodation module 20d, the left part of the user's body portion (for example, the body portion 72 shown in FIG. 14), and the electrode 23 of the accommodation module 20c and then back to the host 10. Alternatively, in some embodiments, the control circuit 63 is configured as a sixth mode: U11 (turned off), U12 (turned on), U13 (turned off), U14 (turned on), U15 (turned off), U16 (turned on), and the current sequentially passes through the electrode 23 of the accommodation module 20b, the right part of the user's body portion (for example, the body portion 71 shown in FIG. 14), the electrode 23 of the accommodation module 20a, the electrode 23 of the accommodation module 20c, the left part of the user's body portion (for example, the body portion 72 shown in FIG. 14), and the electrode 23 of the accommodation module 20d and then back to the host 10. In some other embodiments, the fifth mode may comprise the first mode and the third mode which are alternately configured, and the sixth mode may comprise the second mode and the fourth mode which are alternately configured; for example, each of the modes is output with 50% duty cycle. In some embodiments, the fifth mode and the sixth mode are alternately configured to prevent the risks of electric shock or chemical burning caused by charge accumulation. Moreover, through the configuration of the control circuit 63, under the dual-side therapy mode, the current does not transmit from the electrode 23 contacted by user's right side (left side) to the electrode 23 contacted by user's left side (right side), and the current loop is limited within the end portion of the limb (the palm or sole of the user), thereby preventing current from passing through the user's heart.

In the first mode to sixth mode, each of the electrodes 23 adopts the same current source, therefore the value of the current applied to the body portion can be kept identical. Moreover, in this embodiment, the constant current source element 632 is arranged between the improved bridge circuit and the ground GND as a current sink to limit the constant current passing through the user's body portion; the voltage source VBUS adjusts the value of the voltage according to the resistance change of the user's body portion, thereby maintaining the current to be output stably. In some other embodiments, the constant current source element 632 is arranged between the voltage source VBUS and the improved bridge circuit as a current source. According to some embodiments, an advantage for the given current is that, the user can set the current under the one-side therapy mode; for example, the user can rotate the rotation plate 17 with one hand and place the other hand between the accommodation modules 20a, 20b to feel whether the output current is comfortable or not. After the current is set, under the dual-side therapy mode, a constant current can be applied to the user's two limbs regardless the resistance differences between the two limbs. However, the instant disclosure is not limited to exclude the voltage source as an output supply; for example, in some embodiments, the constant current source element 632 is omitted, and the voltage source VBUS is taken as the constant voltage source.

The method S10 further comprises a step S14, determining whether the user triggers the switch mechanism 13. As shown in FIG. 14, when the user performs iontophoresis therapy on two parts of the user's body portion under the dual-side therapy mode, the body portion 71 (for example, the user's right hand) is placed across the joint portion between the accommodation modules 20a, 20b, and the body portion 72 (for example, the user's left hand) is placed across the joint portion between the accommodation modules 20c, 20d. Specifically, in some embodiments, the rear part 711 of the body portion 71 (for example, the user's right palm) and the rear part 721 of the body portion 72 (for example, the user's left palm) are respectively placed in the accommodation modules 20b, 20d, while the front part 712 of the body portion 71 (for example, the user's right fingers) and the front part 722 of the body portion 72 (for example, the user's left fingers) are respectively placed in the accommodation modules 20a, 20c. Moreover, the support part 713 of the body portion 71 of the user (for example, the user's right wrist) leans against the rear-end plate 214 of the accommodation module 20b, and the support part 723 of the body portion 72 of the user (for example, the user's left wrist) leans against the rear-end plate 214 of the accommodation module 20d.

When the user needs to stop the therapy owing to some reasons, the user can touch the switch mechanism 13 to allow the host 10 to stop outputting current (the step S15). For example, as shown in FIG. 14, the user can use his/her operation portion 714 (for example, the user's thumb) to trigger the switch mechanism 13. In response to that the switch mechanism 13 is triggered, the switch mechanism 13 transmits a trigger signal to the processor 64 (as shown in FIG. 11), and the processor 64 gradually reduces the value of the current outputting to the control circuit 63 to a predetermined current value according to the trigger signal so as to pause the iontophoresis procedure. The predetermined current value may be a predetermined absolute value, for example, a predetermined safety current value which is not relevant to value of the current currently output by the host 10. In one embodiment, the predetermined current value is 0 mA. The current reduction speed may be in the range between 0.5 mA/s and 1 mA/s; by slowly releasing the charges accumulated within the apparatus during the iontophoresis therapy, the user can be prevented from suffering transient electric shock. According to some embodiments, because of the configuration of the switch mechanism 13, when the user's two hands are applied with the therapy, the user's two hands do not need to leave the accommodation modules 20a, 20b, 20c, 20d while the switch mechanism 13 can be stilled triggered. Hence, the issue that the user gets electric shock when the user' body portion leaves the tank occurred to the apparatus known to the inventor can be addressed. Moreover, the front-end plates 213 of the accommodation modules 20a, 20b are between the two host contacts 15 on the first side surface 111 of the host 10, and through the offset configuration of the switch mechanism 13 with respect to the middle point of the two host contacts 15, the switch mechanism 13 is arranged closer to the user's operating portion 714, so that the user 7 can operate the switch mechanism 13 conveniently. According to some embodiments, one advantage of such configuration is that, the user 7 just needs to slightly move the operation portion 714 to operate the switch mechanism 13, while rest parts of the user's body portion can be retained at the original position. It is understood that, during the therapy, as along as the surface of the rear part 711 of the user 7 is moved or lifted with respect to the accommodation modules 20a, 20b or the fabric 30a, discharge may occur; such issue occurs much easily when the amount of the liquid accommodated in the accommodation modules 20a, 20b is less or when the amount of the liquid contained in the fabric 30a is less. Accordingly, in one or some embodiments, through the configuration mentioned above, the risk of electric shock can be effectively reduced while the therapy can be performed with less liquid.

Likewise, when the user tends to start the iontophoresis therapy, the user may allow the host 10 to start outputting current by touching the switch mechanism 13. At this moment, the processor 64 gradually increases the value of the current outputting to control circuit 63 according to the trigger signal to start the iontophoresis procedure. In some embodiments, the step of setting the value of the current of the iontophoresis procedure is executed between the step S12 and the step S13. The user can set the value of the current of the iontophoresis procedure by rotating the rotation plate 17, and the user can start the step S13 by triggering the switch mechanism 13.

Please refer to FIG. 13. In the step S14, in response to that the host 10 determines that the switch mechanism 13 is not triggered, the host 10 continuously outputs current according to the current supply mode (step S16) so as to keep performing the iontophoresis procedure. In the step S17, the host 10 determines whether a scheduled operation time has been reached. If not, the host 10 repeats the step S14; if yes, the host 10 stops outputting current (step S18). In some embodiments, in the step S18, the processor 64 gradually reduces the current output by the control circuit 63 to stop the iontophoresis procedure. The current reduction speed may be in the range between 0.5 mA/s and 1 mA/s, so that the user can be prevented from suffering transient electric shock.

One embodiment of the instant disclosure provides an iontophoresis apparatus comprising two sets of accommodation modules which can be detached from the host. When any set of the accommodation modules is connected to the host, the iontophoresis procedure can be performed, thus addressing the issue that the user cannot perform a one-hand/foot therapy. Moreover, according to one embodiment of the instant disclosure, the accommodation modules and the host are combinable in a novel and innovative manner, and the accommodation modules and the host can be assembled quickly and stored easily. Moreover, according to some embodiments, through a unique control method accompanied with an innovative circuit design, not only the issue of suffering electric shock caused by charge accumulation which cannot be properly addressed in a clinical aspect can be addressed, but also the user is allowed to perform the therapy with fewer amount of liquid, thereby improving the deionization efficiency of the liquid and promoting the performance of the iontophoresis. Furthermore, according to some embodiments, the design of the accommodation modules is ergonomic, thereby greatly improving user's comfortableness.

While the instant disclosure has been described by the way of example and in terms of the preferred embodiments, it is to be understood that the invention need not be limited to the disclosed embodiments. On the contrary, it is intended to cover various modifications and similar arrangements included within the spirit and scope of the appended claims, the scope of which should be accorded the broadest interpretation so as to encompass all such modifications and similar structures.

What is claimed is:

1. An iontophoresis apparatus comprising:
a host comprising:
a housing having a top surface and a first side surface adjacent to the top surface;
a switch mechanism connected to the housing; and
at least two host contacts arranged on the first side surface;
at least two accommodation modules each comprising:
a tank body having two side plates arranged oppositely and a front-end plate connected between the two side plates, wherein a recess is defined in the tank body;
an electrode arranged in the recess of the tank body; and
a tank contact arranged on one of the two side plates and electrically connected to the electrode, wherein the at least two front-end plates of the at least two tank bodies are adapted to be coupled with each other, and the at least two accommodation modules are adapted to be coupled to the host through a connection between the at least two tank contacts and the at least two host contacts; and
a processor configured to change a value of a current transmitted between the host and the at least two electrodes in response to that the switch mechanism is triggered.

2. The iontophoresis apparatus according to claim 1, wherein the switch mechanism is arranged on the first side surface, and a distance between a top surface of the housing and the switch mechanism is less than a distance between the top surface of the housing and each of the at least two host contacts, and a distance between the switch mechanism and a bottom surface of the host is greater than a height of the two side plates of the tank body.

3. The iontophoresis apparatus according to claim 1, wherein the processor is configured to gradually reduce the value of the current transmitted to the two electrodes to a predetermined value of the current in response to that the processor receives a trigger signal from the switch mechanism.

4. The iontophoresis apparatus according to claim 1, further comprising a conductive medium, wherein the conductive medium comprises a first absorbent section, a nonabsorbent section, and a second absorbent section arranged in sequentially, the conductive medium is adapted to be arranged across the two tank bodies, the first absorbent section and the second absorbent section are adapted to respectively cover the two electrodes of the two tank bodies, and the nonabsorbent section extends along the two front-end plates of the two tank bodies.

5. The iontophoresis apparatus according to claim 1, wherein the front-end plate of each of the two tank bodies has a protrusion and an opening, and the two protrusions of the two tank bodies are adapted to be detachably engaged with the two openings of the two tank bodies.

6. The iontophoresis apparatus according to claim 1, wherein for each of the two accommodation modules, a bottom surface of the recess of the tank body at least comprises an inclination section, and a height of the inclination section gradually reduces along a direction away from the front-end plate.

7. The iontophoresis apparatus according to claim 1, wherein the two host contacts are two first magnetic terminal connectors, the two tank contacts are second magnetic terminal connectors, and the two host contacts are attached to the two tank contacts through magnetic forces.

8. The iontophoresis apparatus according to claim 1, wherein the host further comprises a detection circuit, and the detection circuit is configured to determine a connection state between the tank contacts and the two host contacts.

9. The iontophoresis apparatus according to claim 1, wherein the housing of the host further has a second side surface opposite to the first side surface, and the two accommodation modules are separable with each other; in response to that the two tank bodies of the two accommodation modules are stacked with the housing of the host, the first side surface and the second side surface of the host are respectively coplanar with the side plates of the two accommodation modules which are stacked with each other.

10. An iontophoresis apparatus comprising:
a host comprising:
a housing having a top surface and a first side surface adjacent to the top surface;
a switch mechanism connected to the housing; and
at least one host contact arranged on the first side surface;
at least one accommodation tank having two side plates arranged oppositely and an intermediate plate connected between the two side plates, wherein the at least one accommodation tank comprises:
two accommodation modules connected to each other through the intermediate plate, wherein each of the two accommodation modules comprises:
a tank body, wherein a recess is defined in the tank body; and
an electrode arranged in the recess of the tank body; and
a tank contact arranged on one of the two side plates of the accommodation tank and electrically connected to the two electrodes, wherein the accommodation tank is adapted to be coupled to the host through a connection between the tank contact and the host contact; and
a processor configured to change a value of a current transmitted between the host and the two electrodes in response to that the switch mechanism is triggered.

11. An accommodation module comprising:
a tank body having two side plates arranged oppositely and a front-end plate connected between the two side plates, wherein a recess is defined in the tank body, a bottom surface of the recess at least comprises an inclination section, and a height of the inclination section gradually reduces along a direction away from the front-end plate;
an electrode arranged in the recess of the tank body; and
a tank contact arranged on one of the two side plates and electrically connected to the electrode.

12. The accommodation module according to claim 11, wherein the tank contact comprises a magnetic terminal connector.

13. The accommodation module according to claim 11, wherein a gap between the electrode and the tank body is sealed.

14. The accommodation module according to claim 11, further comprising a connection structure, wherein the connection structure is configured to allow the accommodation module to be detachably connected to another accommodation module.

15. A method for operating control circuit, wherein the method comprises:
executing a connection state determination operation, wherein the connection state determination operation comprises:
turning on a first switch;
transmitting a test signal from a test signal source to a first host contact through the first switch which is turned on; and
receiving the test signal from the first host contact by using a detection circuit to determine whether the first host contact is coupled to a first tank contact.

16. The method for operating control circuit according to claim 15, wherein the connection state determination operation further comprises:
turning on the first switch and a second switch, wherein the second switch is connected in series to a downstream of the first switch;
transmitting another test signal from the test signal source to a second host contact through the first switch and the second switch which are turned on; and
receiving the another test signal from the second host contact by using the detection circuit to determine whether the second host contact is coupled to a second tank contact.

17. The method for operating control circuit according to claim 15, further comprises:
executing an error determination operation before executing the connection state determination operation, wherein the error determination operation comprises:
turning on a second switch and a third switch, wherein the second switch and the third switch are sequentially connected in series to a downstream of the first switch; and
under a condition that the second switch and the third switch are kept turned on, adjusting the first switch from a turned-off state to a turned-on state, and receiving the test signal from the test signal source by using a current measurement element to determine whether the first switch functions normally.
